(19) United States
(12) Patent Application Publication
(10) Pub. No.: US 2007/0048362 A1
(43) Pub. Date: Mar. 1, 2007

(54) General purpose electrolyte solution composition for iontophoresis

(75) Inventors: Mizuo Nakayama, Shibuya-ku (JP); Takehiko Matsumura, Shibuya-ku (JP); Hidero Akiyama, Shibuya-ku (JP); Akihiko Matsumura, Shibuya-ku (JP)

Correspondence Address: SEED INTELLECTUAL PROPERTY LAW GROUP PLLC, 701 FIFTH AVE, SUITE 5400, SEATTLE, WA, 98104, US

(73) Assignee: Transcutaneous Technologies Inc., Shibuya-ku (JP)

(21) Appl. No.: 11/512,756

(22) Filed: Aug. 29, 2006

Related U.S. Application Data
(60) Provisional application No. 60/720,822, filed on Sep. 26, 2005.

(30) Foreign Application Priority Data
Aug. 29, 2005 (JP) 2005-247994

(51) Int. Cl. A61N 1/30; A61F 13/00

(52) U.S. Cl. 424/449; 604/20

(57) ABSTRACT

A single electrolyte solution composition may be used in an anode side and in a cathode side of an iontophoresis device. The electrolyte solution may include a compound having an oxidation-reduction potential lower than that of water, the compound including, in combination, both a component that is likely to be relatively reduced and a component that is likely to be relatively oxidized.

### CROSS-REFERENCE TO RELATED APPLICATION

This application claims priority from Japanese Application No. 2005-247994, filed Aug. 29, 2005, now pending, which application is incorporated herein by reference in its entirety. This application also claims the benefit under 35 U.S.C. § 119(E) of U.S. Provisional Patent Application No. 60/720,822, filed Sep. 26, 2005, which application is incorporated herein by reference in its entirety.

### BACKGROUND

1. Field

The present disclosure generally relates to the field of transdermal delivery of an ionic active agent via iontophoresis, and in particular, to an electrolyte solution composition that may be used as a conductive medium in an iontophoresis device.

2. Description of the Related Art

Iontophoresis (also known as iontophorese, ion introduction method, ion permeation therapy; see JP 63-35266 A) is a method of delivering an ionic active agent placed on biological interface, mucosa, or other biological interface into a subject by means of an electromotive force sufficient to drive the ionic active agent into and/or through the biological interface.

For example, positively charged ions may be delivered into the biological interface from an anode (positive electrode) of an iontophoresis device, while negatively charged ions may be delivered into the biological interface from a cathode (negative electrode) thereof.

Device structures and constituent materials for iontophoresis have already been proposed (for examples, refer to JP 63-35266 A, JP 04-297277 A, JP 2000-229128 A, JP 2000-229129 A, JP 2000-237327 A, JP 2000-237328 A, and/or WO 03/037425 A1).

Physiological saline (an aqueous solution of NaCl) has conventionally often been used as a conductive medium in an electrode assembly of an iontophoresis device. However, an electrochemical reaction may occur on an anode (positive electrode) side and a cathode (negative electrode) side when using physiological saline, leading to the electrolytic reaction of an electrolyte solution. As a result, gas bubbles may be generated at both electrodes. For example, hydrogen gas may be generated at the cathode, while chlorine gas and oxygen gas may be generated at the anode. Gas bubbles may significantly increase the electrical resistance of electrode surfaces, inhibiting the flow of electric current.

One method that may be used to reduce the likelihood of gas generation is to add a compound to the electrolyte that is likely to be oxidized or reduced at an electric potential lower than that required for an electrolytic reaction of water (oxidation at the anode or reduction the cathode). Refer to JP 2000-229128 A for an example of such. Ferrous sulfate, ferric sulfate, or an organic acid having an oxidation-reduction potential lower than the electrolytic potential of water may be used. Specifically, a conductive medium on a cathode side of an iontophoresis electrode assembly may comprise physiological saline containing a compound that is likely to be reduced (ferric sulfate), while a conductive medium on an anode side of the iontophoresis electrode assembly may comprise physiological saline containing a compound that is likely to be oxidized (ferrous sulfate). When ferric sulfate is used, ferric ions may be reduced to ferrous ions at the cathode. When ferrous sulfate is used, ferrous ions may be oxidized to ferric ions at an anode. Problems relating to the generation of gas due to the electrolytic reaction of water may thus be mitigated.

In such a device, however, appropriate compositions may need to be prepared for each electrode (cathode and anode), thus complicating iontophoresis device production. Costs may increase, and a suitable electrolyte solution must be identified for each electrode. This may be disadvantageous to handling.

### BRIEF SUMMARY OF THE INVENTION

In one aspect, the present disclosure is directed to a general purpose electrolyte solution composition capable of being used as a conductive medium in an anode and in a cathode of an iontophoretic device. The electrolyte solution composition may include a compound having an oxidation-reduction potential lower than that of water. The compound may contain, in combination, a component that is likely to be relatively reduced and a component that is likely to be relatively oxidized relative to water.

In one aspect, the present disclosure is directed to a general purpose electrolyte solution composition that may be prepared by adding a component that oxidizes at an electrical potential lower than that the oxidation potential of water on an anode side, and a component that reduces at an electrical potential higher than the reduction potential of water on a cathode side in combination.

In one aspect, the present disclosure is directed to an electrolyte solution composition which may:
(A) include a buffer action; and/or
(B) cause substantially no chemical reactions between components when not being used or during storage; and/or
(C) be an aqueous solution containing three or more components in a composite manner; and/or
(D) be substantially harmless to a human body.

In one aspect, the present disclosure is directed to a general purpose electrolyte solution composition that may include an ascorbate and a fumarate.

In one aspect, the present disclosure is directed to a general purpose electrolyte solution composition that further contains polyacrylic acid and/or lactic acid to impart a buffering action.

In one aspect, the present disclosure is directed to a gel matrix that includes a gel containing the above electrolyte solution composition.

In one aspect, the present disclosure is directed to an electrode assembly for an iontophoresis device that includes any of the electrolyte solutions described above.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 shows an iontophoresis device according to one illustrated embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with iontophoresis devices, controllers, voltage or current sources and/or membranes have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “another embodiment” means that a particular referent feature, structure, or characteristic described in connection with the embodiment is included in at least one embodiment. Thus, the appearances of the phrases “in one embodiment,” or “in an embodiment,” or “another embodiment” in various places throughout this specification are not necessarily all referring to the same embodiment. Further more, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to a system for evaluating an iontophoretic active agent delivery device including “a controller” includes a single controller, or two or more controllers. It should also be noted that the term “or” is generally employed in its sense including “and/or” unless the content clearly dictates otherwise.

As used herein the term “membrane” means a boundary, a layer, barrier, or material, which may, or may not be permeable. The term “membrane” may further refer to an interface. Unless specified otherwise, membranes may take the form a solid, liquid, or gel, and may or may not have a distinct lattice, non cross-linked structure, or cross-linked structure.

As used herein the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH and ACS also from Tokuyama Co., Ltd.

As used herein, the term bipolar membrane means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two film structure) may include a cation exchange membrane laminated or otherwise electrically coupled to an anion exchange membrane. The cation and anion exchange membranes initially start as distinct structures, and may or may not retain their distinctiveness in the structure of the resulting bipolar membrane.

As used herein, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules a first rate, and some other molecules a second rate different than the first. In yet further embodiments, the “semi-permeable membrane” may take the form of a selectively permeable membrane allowing only certain selective molecules to pass through it.

As used herein, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles). In some embodiment a gel matrix may include hydrogels, organogels, and the like. Hydrogels refer to three-dimensional network of, for example, cross-linked hydrophilic polymers in the form of a gel and substantially composed of water. Hydrogels may have a net positive or negative charge, or may be neutral.

A used herein, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

A used herein, the term “active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., an active agent, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent. In some embodiments, the term “active agent” further refers to the active agent, as well as its pharmacologically active salts, pharmaceutically acceptable salts, prodrugs, metabolites, analogs, and the like. In some further embodiment, the active agent includes at least one ionic, cationic, ionizable and/or neutral therapeutic active agent and/or pharmaceutical acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. While other active agents may be polarized or polarizable, that is exhibiting a polarity at one portion relative to another portion. For instance, an active agent having an amino group can typically take the form an ammonium salt in solid state and dissociates into a free ammonium ion (NH₄⁺) in an aqueous medium of appropriate pH. The term “active agent” may also refer to neutral agents, molecules, or compounds capable of being delivered via electro-osmotic flow. The neutral agents are typically carried by the flow of, for example, a solvent during electrophoresis. Selection of the suitable active agents is therefore within the knowledge of one skilled in the art.

Non-limiting examples of such active agents include lidocaine, articaine, and others of the -caine class; morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate and other 5-hydroxytryptaminel receptor subtype agonists; resiquimod, imiquidmod, and similar TLR 7 and 8 agonists and antagonists; domperidone, granisetron hydrochloride, ondansetron and such anti-emetic active agents; zolpidem tartrate and similar sleep inducing agents; L-dopa and other anti-Parkinson's medications; aripiprazole, olanzapine, quetiapine, risperidone, clozapine and ziprasidone as well as other neuroleptica; diabetes active agents such as exenatide; as well as peptides and proteins for treatment of obesity and other maladies.

As used herein and in the claims, the term “subject” generally refers to any host, animal, vertebrate, or invertebrate, and includes fish, mammals, amphibians, reptiles, birds, and particularly humans.

The headings provided herein are for convenience only and do not interpret the scope or meaning of the embodiments.

Iontophoresis Device

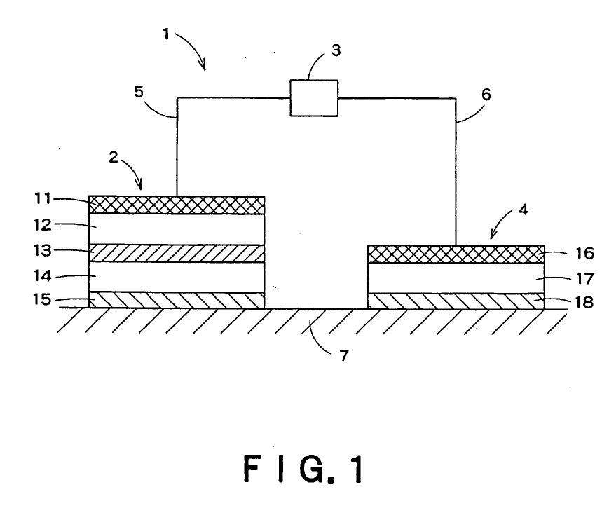

FIG. 1 shows an iontophoresis device 1 comprising an active electrode assembly 2 is placed on the surface of a biological interface 7. The iontophoresis device 1 further comprises an electric power source device 3, and a counter electrode assembly 4. The active electrode assembly 2 comprises: an electrode 11 connected via a lead wire 5 to the electric power source device 3 that imparts an electric potential having the same polarity as that of a charged ion of an active agent; an electrolyte solution reservoir 12 that holds an electrolyte solution, the electrolyte solution reservoir 12 electrically coupled to the electrode 12; an ion exchange membrane 13 that selectively passes ions having a polarity opposite to that of active agent ions, the ion exchange membrane 13 electrically coupled to the electrolyte solution reservoir 12; an active agent solution reservoir 14 that holds the ionic active agent, the active agent solution reservoir 14 electrically coupled to the ion exchange membrane 13; and an ion exchange membrane 15 that selectively passes ions having the same polarity as that of the active agent ions, the ion exchange membrane 15 electrically coupled to the active agent solution reservoir 24.

The counter electrode assembly 4 comprises: an electrode 16 having a polarity opposite to that of the electrode 11 of the active electrode assembly 2, the electrode 16 being electrically coupled via a lead wire 6 to the electric power source device 3; an electrolyte solution reservoir 17 that holds an electrolyte solution, the electrolyte solution reservoir 17 electrically coupled to the electrode 16; and an ion exchange membrane 18 that selectively passes ions having a polarity opposite to that of the charged ion of the ionic active agent, the ion exchange membrane 18 electrically coupled to the electrolyte solution reservoir 17.

Upon energization, the ionic active agent migrates away from the electrode 11 by virtue of an electric field, and is efficiently released through the ion exchange membrane 15. The ionic active agent may efficiently be administered to a biological interface 7 because substantially no competitive ionic species are present in the active agent solution reservoir 14. Examples of competitive ionic species include un-reacted cross linking agents, cross linking initiators, and monomers.

The electrolyte solution described here may be commonly used in both the active electrode assembly and in the counter electrode assembly described above. The composition of the electrolyte solution composition is described in detail below.

Conditions under which the active agent solution reservoir 14 is impregnated with an ionic active agent and an electrolyte solution may be suitably determined in accordance with, for example, impregnation amounts and impregnation speeds of the ionic active agent and the electrolyte solution. Example conditions for impregnation include a temperature of 40° C. and a time period of 30 minutes.

The active agent solution reservoir 14 may comprise a thin film. The active agent solution reservoir 14 may be used as, for example, an ion conductive porous sheet for forming a gel-like solid electrolyte in accordance with the description of JP 11-273452 A.

A cation exchange membrane and an anion exchange membrane may be advantageously used together as the ion exchange membrane 13 and the ion exchange membrane 15. The electrolyte solution reservoir 12 may comprise a thin film capable of holding an electrolyte solution.

In addition, an inactive electrode made of a conductive material such as carbon or platinum may be used as the electrode in one or both of the electrode assemblies.

WO 03/037425 A1, incorporated herein by reference in its entirety, describes components that may be used in the iontophoresis device 1.

Electrolyte Solution Composition

An electrolyte solution composition may be used as a conductive medium of an iontophoresis device in both an anode and a cathode. The electrolyte solution may comprise a compound having an oxidation-reduction potential lower than that of water, the compound comprising, in combination, a component that is likely to be relatively reduced and a component that is likely to be relatively oxidized.

Specific examples of such components include a combination of an ascorbate and a fumarate. Those compounds effect substantially no chemical reaction in aqueous solution states in a typical condition (when not being used, during storage) when used in combination.

Examples of ascorbates include trisodium ascorbyl2-phosphate, magnesium ascorbate, and disodium ascorbyl2-phosphate.

Examples of fumarates that may be used include sodium fumarate and potassium fumarate.

Ascorbates may effectively inhibit the electrolysis of water at an anode because it is oxidized at an electric potential lower than the oxidation potential of water. In addition, electrolysis of water may not occur at a cathode because the above-described fumarate is reduced at an electric potential higher than the reduction potential of water. The generation of gas resulting from the electrolysis of water may thus be inhibited.

A buffering agent, such as polyacrylic acid or lactic acid, may also be used in the electrolyte solution in order to stabilize pH.

The general purpose electrolyte solution composition may be the form of a gel matrix containing the composition.

### EXAMPLE

An iontophoresis device having the configuration shown in FIG. 1 was tested. An electrolyte solution composition having the following composition was applied to the electrolyte solution reservoir 12 of the active electrode assembly 2 and the electrolyte solution reservoir 17 of the counter electrode assembly.

### Composition of General Purpose Electrolyte Solution Composition

| Components | Molar concentration |
|---|---|
| Trisodium ascorbyl2-phosphate | 0.42 M |
| Sodium fumarate | 0.019 M |
| Polyacrylic acid (molecular | 0.139 M |
| weight: 25,000) | |
| Water | Balance |

Energization Test

An electrolyte solution reservoir holding the general purpose electrolyte solution shown above was mounted on each of the active electrode assembly and the counter electrode assembly, and then an active agent release test was performed under the following conditions. The generation of gas due to the electrolysis of water was not observed.

Drug solution reservoir used: Lidocaine (2%)

Electrode: Carbon

Energizing conditions: 0.94 mA/cm2, 90 minutes

The above description of illustrated embodiments, including what is described in the Abstract, is not intended to be exhaustive or to limit the embodiments to the precise forms disclosed. Although specific embodiments and examples are described herein for illustrative purposes, various equivalent modifications can be made without departing from the spirit and scope of the disclosure, as will be recognized by those skilled in the relevant art. The teachings provided herein of the various embodiments can be applied to other problem-solving systems devices, and methods, not necessarily the exemplary problem-solving systems devices, and methods generally described above.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety.

Aspects of the embodiments can be modified, if necessary, to employ systems, circuits, and concepts of the various patents, applications, and publications to provide yet further embodiments.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

These and other changes can be made to the embodiments in light of the above-detailed description. In general, in the following claims, the terms used should not be construed to limit the invention to the specific embodiments disclosed in the specification and the claims, but should be construed to include all possible embodiments along with the full scope of equivalents to which such claims are entitled. Accordingly, the scope of the invention shall only be construed and defined by the scope of the appended claims.

What is claimed is:

1. A general purpose electrolyte solution for an iontophoresis device, comprising:
a compound having an oxidation-reduction potential lower than that of water, the compound including, in combination, a component likely to be relatively reduced and a component likely to be relatively oxidized.

2. The general purpose electrolyte solution composition according to claim 1, further comprising polyacrylic acid and/or lactic acid as a component that imparts a buffering action.

3. The general purpose electrolyte solution composition according to claim 1 wherein the compound comprises a component that oxidizes at a potential lower than the oxidation potential of water, and a component that reduces at a potential higher than the reduction potential of water are included in combination.

4. The general purpose electrolyte solution composition according to claim 3, further comprising polyacrylic acid and/or lactic acid as a component that imparts a buffering action.

5. The general purpose electrolyte solution composition according to claim 1, further comprising:
a component that imparts a buffering action;
wherein no substantial chemical reaction occurs between the components when an electric potential is not applied to the general purpose electrolyte solution; and
wherein the composition causes substantially no harm to a subject.

6. The general purpose electrolyte solution composition according to claim 5, further comprising polyacrylic acid and/or lactic acid as a component that imparts a buffering action.

7. The general purpose electrolyte solution composition according to claim 1 wherein the component likely to be relatively oxidized comprises an ascorbate, and the component likely to be relatively reduced comprises a fumarate.

8. The general purpose electrolyte solution composition according to claim 7, further comprising polyacrylic acid and/or lactic acid as a component that imparts a buffering action.

9. A gel matrix composition comprising a gel that includes a compound having an oxidation-reduction potential lower than that of water, the compound including, in combination, a component likely to be relatively reduced and a component likely to be relatively oxidized.

10. The gel matrix composition according to claim 9, further comprising polyacrylic acid and/or lactic acid as a component that imparts a buffering action.

11. The gel matrix composition according to claim 9, further comprising:
a component that imparts a buffering action;
wherein no substantial chemical reaction occurs between the components when an electric potential is not applied to the general purpose electrolyte solution; and
wherein the composition causes substantially no harm to a subject.

12. An iontophoresis device, comprising:
an electrode assembly that includes a compound having an oxidation-reduction potential lower than that of water, the compound including, in combination, a component likely to be relatively reduced and a component likely to be relatively oxidized.

13. The iontophoresis device according to claim 12 wherein the electrode assembly further comprises polyacrylic acid and/or lactic acid as a component that imparts a buffering action.

14. The gel matrix composition according to claim 9 wherein the electrode assembly further comprises a component that imparts a buffering action wherein no substantial chemical reaction occurs between the components when an electric potential is not applied to the general purpose electrolyte solution; and wherein the composition causes substantially no harm to a subject.

15. The iontophoresis device according to claim 12 wherein the electrode assembly is an active electrode assembly comprising an active agent.

16. The iontophoresis device according to claim 12 wherein the electrode assembly is a counter electrode assembly.

17. The iontophoresis device according to claim 12, further comprising:
a power source coupled to the electrode assembly.
